SEQUENCE LISTING (1 sequence)

SEQ ID NO: 1 - DNA, 1645 bp, Scenedesmus species Novo
  ref <308>: DATABASE ACCESSION NUMBER: Genbank HQ900842
  ref <309>: DATABASE ENTRY DATE: 2011-03-06
  ref <313>: RELEVANT RESIDUES IN SEQ ID NO: (1)..(1645)
tgcttatactgtgaaactgcgaatggctcattaaatcagttatagtttatttggtggtaccttcttactcggaataaccgtaagaaaattagagctaatacgtgcgtaaatcccgacttctggaagggacgtatatattagataaaaggccgaccgggctctgcccgacccgcggtgaatcatgatatcttcacgaagcgcatggccttgtgccggcgctgttccattcaaatttctgccctatcaactttcgatggtaggatagaggcctaccatggtggtaacgggtgacggaggattagggttcgattccggagagggagcctgagaaacggctaccacatccaaggaaggcagcaggcgcgcaaattacccaatcctgatacggggaggtagtgacaataaataacaataccgggcatttcatgtctggtaattggaatgagtacaatctaaatcccttaacgaggatccattggagggcaagtctggtgccagcagccgcggtaattccagctccaatagcgtatatttaagttgttgcagttaaaaagctcgtagttggatttcgggtgggtttcagcggtccgcctatggtgagtactgctgtggccttccttactgtcggggacctgcttctggggcttcattgtccgggacagggattcggcatggttactttgagtaaattagagtgttcaaagcaggcttacgcccgtgaatactttagcatggaataacatgataggactctgccctattctgttggcctgtaggagtggagtaatgattaagaggaacagtcgggggcattcgtatttcattgtcagaggtgaaattcttggatttatgaaagacgaactactgcgaaagcatttgccaaggatgttttcattaatcaagaacgaaagttgggggctcgaagacgattagataccgtcgtagtctcaaccataaacgatgccgactagggattggcggacgtttttgcatgactccgtcagcaccttgagagaaatcaaagtttttgggttccggggggagtatggtcgcaaggctgaaacttaaaggaattgacggaagggcaccaccaggcgtggagcctgcggcttaatttgactcaacacgggaaaacttaccaggtccagacataggaaggattgacagattgagagctctttcttgattctatgggtggtggtgcatggccgttcttagttggtgggttgtcttgtcaggttgattccggtaacgaacgagacctcagcctttaaatagtcacggtcgctttttgcggctggtctgacttcttagagggacagttggcgtttagtcaacggaagtatgaggcaataacaggtctgtgatgcccttagatgttctgggccgcacgcgcgctacactgatgcattcaacaagcctatccctagccgaaaggctcgggtaatctttgaaactgcatcgtgatggggatagattattgcaattattagtcttcaacgaggaatgcctagtaagcgcaattcatcagattgcgttgattacgtccctgccctttgtacacaccgcccgtcgctcctaccgattgggtgtgctggtgaagtgttcggattggcaattgaaggtggcaacaccgtcgatgccg

(19) United States
(12) Patent Application Publication
(10) Pub. No.: US 2013/0164322 A1
(43) Pub. Date: Jun. 27, 2013

(54) CULTURED EXTREMOPHILIC ALGAE SPECIES NATIVE TO NEW MEXICO

(71) Applicants: University Of New Mexico, Albuquerque, NM (US); Department Of Veterans Affairs, Washington, DC (US)

(72) Inventors: Ravi Venkata Durvasula, Albuquerque, NM (US); Subba Rao Durvasula, Dartmouth (CA); Annabeth Fieck, Albuquerque, NM (US); Ivy Hurwitz, Albuquerque, NM (US)

(73) Assignees: DEPARTMENT OF VETERANS AFFAIRS, Washington, DC (US); UNIVERSITY OF NEW MEXICO, Albuquerque, NM (US)

(21) Appl. No.: 13/723,687

(22) Filed: Dec. 21, 2012

Related U.S. Application Data
(60) Provisional application No. 61/579,120, filed on Dec. 22, 2011.

(51) Int. Cl. C12N 1/12

(52) U.S. Cl. CPC C12N 1/12
USPC 424/195.17; 435/257.5

(57) ABSTRACT

Provided herein is an extremophile green alga designated as Scenedesmus species Novo, from Jemez warm water springs, New Mexico. Sequencing 18S rDNA confirmed the alga as a new species. It is capable of producing high levels of microalgal biomass in wastewater under harsh ambient climatic conditions, and of yielding high levels of lipids and carotenes. Cultures in TAP medium at 24±1° C. at continuous light (132-148 μmol photons m⁻²s⁻¹) attained peak biomass levels 27.4×10⁶ cells ml⁻¹, 49.11 μg ml⁻¹ chlorophyll α, 24.93 μg ml⁻¹ carotene on the seventh day and a division rate of 0.54 day⁻¹. High levels of biomass were sustained in sterilized and unsterilized municipal wastewater, enriched with 1% TAP nutrients or unenriched. The microalga is useful in the production of biofuels, fertilizers, dietary nutrients, pharmaceuticals, polymers, biofilters to remove nutrients and other pollutants from wastewaters, in space technology, and laboratory research systems.

### CROSS-REFERENCE TO RELATED APPLICATIONS

This application is a nonprovisional application claiming priority to U.S. Provisional Patent Application Ser. No. 61,579,120 filed Dec. 22, 2011, which is incorporated herein by reference to the extent not inconsistent herewith.

### STATEMENT OF GOVERNMENT SUPPORT

This invention was made at least in part with Government support from the Department of Veterans Affairs. The Government has certain rights in the invention.

### BACKGROUND

Despite significant investment in research and development, commercial viability of algal-derived biofuels remains a future prospect. Costs of mass algal culture, unpredictability of algal stocks, high costs of algal concentration and extraction of products and limited choices for algal stocks all contribute to the untenable costs of algal biofuel—in excess of $17 per gallon—and the limited use of this energy source in the open market.

### SUMMARY

Provided herein is an isolated and purified new microalgal species designated Scenedesmus species Novo and progeny thereof. The alga was collected at latitude 35.769 and longitude 106.692. It is capable in culture including TAP medium of producing a biomass of about 10.41×10⁶ cells per ml and at least about 4 μg per ml, for example, about 4.18 to about 4.5 μg per ml, of carotene under outdoor growth conditions comprising temperatures reaching 40° C. or higher.

The new microalgal species has an 18S ribosomal RNA gene sequence [SEQ ID NO:1] at least about 99% to about 100% identical to SEQ ID NO:1, and about 98% identical to algal species G24 (38).

In embodiments, the alga is capable of producing up to at least about 3.58 μg per cell of carotene under indoor growth conditions. The term “up to at least about” as used with respect to a numerical value herein refers to a value seen at any point on a graph of such values over time.

The cultures can be cultivated in wastewater at temperatures of at least about 40° C. In embodiments the cultures are cultivated at temperatures between about 40° C. and about 100° C., or between about 40° C. and about 80° C., or between about 40° C. and about 60° C., or between about 40° C. and about 50° C. As used herein, the term “extremophilic microalgae” refers to thermophilic microalgae capable of growth at such temperatures.

Cultivated in water enriched with growth-promoting nutrients such as those of TAP medium, at ambient room temperatures (e.g.; about 20° C. to about 26° C.), cultures of this microalga are capable of producing an average lipid content of between about 63 pg per cell and about 95 pg per cell. Cultures grown in enriched TAP medium indoors can have a chlorophyll α content up to between about 20 and about 49 μg per ml, and a carotene content up to about 10 to about 24 or about 25 μg per mi.

In embodiments, grown outdoors in wastewater at temperatures that reach 40° C. or higher, in a TAP medium, such cultures can have a lipid content of between about 16.7 and 81.4 pg per cell, a chlorophyll α content up to about 5.8 μg ml⁻¹, and a carotene content over 4 μg ml⁻¹, e.g., about 4.18 to about 4.5 μg ml⁻¹.

The cultured algae are circular and can be single cells and/or clumps of up to about 360 cells which drop to the bottom of the vessel containing the culture, thus making it easy to harvest the cells. Harvested algal biomass produced by the microalgae can be dried to a mass having a water content less than about 5%.

A method for culturing and harvesting extremophilic microalgae is also provided herein. The method comprises preparing a growth medium composition comprising said extremophilic microalgae and water comprising nutrients capable of enhancing growth of the microalgae; allowing the microalgae to proliferate in the composition under ambient outdoor conditions comprising intervals of ambient temperatures of at least about 40° C. and ambient light of up to about 1400 to about 1600 watts; and dewatering the composition and recovering an drying it to obtain an algal biomass comprising the microalgae and less than about 5% water content.

The dewatering step can be performed in an AlgaeVenture Systems micro-Solid-Liquid-Separation System (http://www.youtube.com/watch?v=CgBMK6DMheQ; http://Algaevs.com), AlgaeVenture Systems, Marysville, Ohio. In embodiments, the extremophilic microalgae in the growth composition are Scenedesmus species Novo. In embodiments, the growth composition also comprises wastewater. In embodiments, the nutrients in the growth composition are selected from the group consisting of TAP medium components, selenium, boron and iron. The wastewater can be sterilized urban or agricultural wastewater or nonsterilized urban or agricultural wastewater.

In another embodiment hereof, a method for culturing and harvesting extremophilic microalgae is provided comprising: preparing a growth composition comprising the extremophilic microalgae and water comprising TAP medium components in amounts sufficient to enhance growth of said microalgae; allowing the microalgae to proliferate in said composition at room temperatures, such as temperatures of about 23° C. to about 25° C.; and dewatering and drying the composition and recovering an algal biomass comprising the microalgae and less than about 5% water content. In embodiments of this method, the microalgae are Scenedesmus species Nova. In embodiments, the growth composition comprises wastewater and TAP medium.

A method of inhibiting growth of a microorganism is also provided herein. The method comprises contacting cells of the microorganism with an extract of Scenedesmus species Nova. The microorganisms can be bacteria, viruses, parasites, or fungi.

Applicants have isolated an extremophile green alga, Scenedesmus species Novo, with unique growth and biochemical characteristics, from Jemez warm water springs in New Mexico. Sequencing 18S rDNA confirmed the alga as a new species. Cultures in TAP medium at 24±1° C. at continuous light (132-148 μmol photons m⁻²s⁻¹) attained peak biomass levels of 27.4×10⁶ cells ml⁻¹ with a division rate (k) of 0.54 day⁻¹, and yielded 49.11 μg chlorophyll α ml⁻¹ and 24.93 μg carotene ml⁻¹ on the 7th day high levels of biomass were sustained in sterilized or unsterilized municipal wastewater, either enriched with 1% TAP nutrients or unenriched. Under outdoor conditions (6524-7360 μmol photons m⁻²s⁻¹ and ˜40° C.), high levels of biomass (10.41×10⁶ cells ml⁻¹), and yields of 8.92 μg chlorophyll α ml⁻¹, and 4.18 μg carotene ml⁻¹ were sustained. Lipids in cells raised in TAP under controlled, less severe conditions ranged from 63 to 94.3 pg cell⁻¹, and in outdoor wastewater 16.7 to 81.4 pg cell⁻¹, which are higher than those previously reported in the literature. In cultures raised in TAP in outdoor waste water, lipid (% of cell dry weight) ranged from 15% to 74%, substantially higher than previous literature values. Total carotenoids ranged between 0.37 and 3.58 pg cell⁻¹.

Because of its ability to produce high levels of microalgal biomass in wastewater under harsh ambient climatic conditions and yield of high levels of lipids and carotenes, mass cultivation of Scenedesmus species Novo is useful in many biotechnological applications.

### BRIEF DESCRIPTION OF THE DRAWINGS

FIG. 1. Con-focal image of Scenedesmus species Nova.

FIG. 2. Growth of Scenedesmus species Novo in TAP and BG11 media.

FIG. 3. Temporal variations in cellular pigments in indoor cultures.

FIG. 4. Temporal variations in cellular pigments in outdoor cultures.

### DETAILED DESCRIPTION

Microalgae have several advantages as feedstock to land-based biofuels. They are renewable and amenable for mass cultivation on nonarable land: they can be a source of significant quantities of lipids: they act as a source of value-added co-products: they can be used for bioremediation; and they are capable of sequestering carbon. Microalgal biomass can yield between 58,700 and 90,000 liters of biodiesel per hectare per year (1, 2, 3). Biofuels contribute to ˜2% of global transport fuel today but are predicted to increase to 27% by the year 2050 (4). For biotechnological applications, sustenance and steady supply of algal biomass are required, which is feasible by mass cultivation of algae. Only a small percentage of the 17,500 microalga species are cultured and about 50 have been screened for their utility—mostly in biofeed, with only a few having been identified as useful for biofuel. Most of the algal isolates are from temperate waters and are grown in defined sterile media under controlled conditions of temperature and light, which collectively escalate biomass production costs to as high as $7.32 per kg of algal biomass and $24.60 per liter algal oil (5).

Microalgae characterized as extremophiles remain least studied. Extremophile algae can readily adapt to exacting local physicochemical conditions, and manifest biochemical and physiological responses such as the production of carotenoids, as in Dunaliella salina (6). The extremophile diatom Nitzschia frustula and the green alga Chiamydomonas plethora isolated from the semiarid harsh climate of the Arabian Gulf (7) have high division rates, carbon assimilation rates (18.1 22.8 to mg C per mg chlorophyll α per hour) approaching their theoretical maxima as well as yielding levels of acids and leucine, lysine, glutamic acid and arginine that make them valuable in biotechnological applications.

Reported here are observations on Scenedesmus species Novo, an extremophile green alga isolated by us from Jemez Springs, N. Mex. This alga grows well in urban wastewater under ambient conditions of light and temperature in New Mexico, yields considerable quantities of lipids and carotenoids and is especially useful for producing algal biofuel.

### EXAMPLES

### Cultured Cells of Scenedesmus Species Novo

Several samples of water were collected from Jemez warm water springs (latitude 35.769 and longitude 106.692) and enriched with nutrients f/50, f/10 (28) and TAP media. Samples were incubated at 24±1° C. at continuous 132-148 μmol m⁻²s⁻¹ light supplied by cool white fluorescent lights. Using repeated serial dilution techniques algal cultures were established. Pure cultures were based on isolates established by streaking on agar plates. Agar slants were based on enrichments with f/50, BG11 and TAP media. Utilizing usual sterile culture techniques, colonies were isolated and gradually scaled up into BG11 (29) and modified TAP medium (30). TAP medium based on enrichment with 10 ml each of triacetate stock, nutrient stock, phosphate buffer and trace elements supported excellent growth. Trace element enrichment follows the formula as described in Hunter (1950) (31).

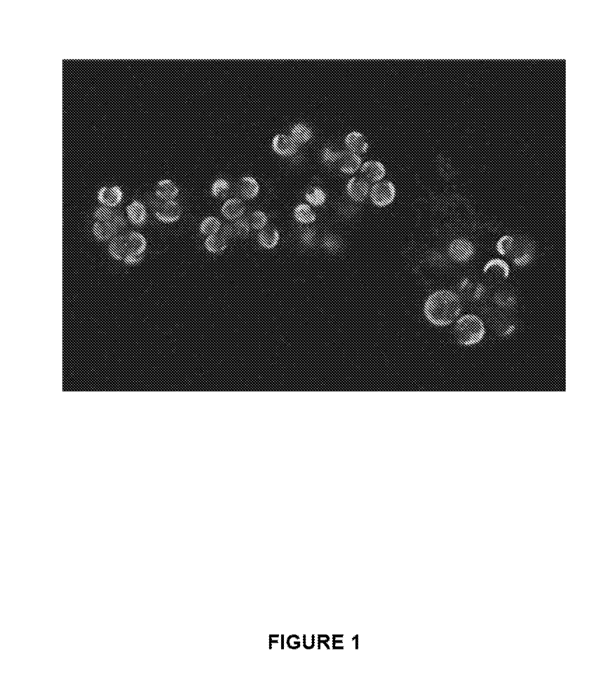

Cultured cells of Scenedesmus species Novo were circular, either singular or in clumps up to 356 cells and did not have any spines. Cells were non-motile, enveloped in mucilage (FIG. 1). Well-mixed cells left in culture flasks sank to the bottom in a couple of minutes which is advantageous in harvesting the biomass.

All algae samples collected from this location exhibited similar properties and were considered to be samples of the same species.

### Cultures in Defined Media

All growth experiments were done in triplicate. Samples were incubated at 24±1° C. at continuous 132-148 μmol m⁻²s⁻¹ light supplied by fluorescent lights, or were incubated over the terrace of a building under natural light (1400-1600 watts m⁻², equivalent to 6524-7360 μmol m⁻²s⁻¹), and ˜40° C. Suitable aliquots were drawn from each culture aseptically for enumeration, chlorophyll a and carotenoid determinations. Direct counts were made on the samples using an Improved Neubauer haemocytometer.

### Division Rates

Based on direct cell counts generative times in hours were calculated (33). The division rate of cells was 0.54 day⁻¹ in TAP medium and 0.27 day⁻¹ in BG 11 (Table 1).

### Chlorophyll α and Carotenoids

For chlorophyll α and carotenoids, a one-ml sample was centrifuged into a pellet and sonicated with a Branson sonicator with a fine probe for one minute at 0° C. in ice cold 90% acetone. The contents were thoroughly mixed in a vortex mixer and extracted for 24 h at 4° C. in a refrigerator sufficient for complete extraction. The extracts were cleared by centrifugation in a Beckman CS 15R centrifuge, and their absorptions at 750 (blank), 664, 647, and 452 nm, were read in a Spectromax spectrofluorimeter that accommodates 96 well polypropylene NUNC plates.

The following equations were used to calculate pigment concentrations (μg ml culture):

Chlα=11.93D664−1.93D647(Vc/Vs) (34)

Carotenoid=3.86*D452(Vc/Vs) (35)

where Vc=volume of culture sample (ml) and Vs=Volume of extract (ml).

Quantitative measurement of fatty acids was performed by Avanti Polar Lipids, Inc. (www.Avantilipids.com) of fatty acid methyl ester (FAME) by gas chromatography with flame ionization (GC/FID) on 1.5 ml of extracted algae using 7-level calibration curves of FAME standards for C8-C24:1 compounds with a C15:1 as internal standard (36). Each sample was injected in triplicate. Standard deviation of the mean ranged between 0.01 and 0.04 when the mean total lipids were <6.0, and between 0.71 and 2.71 when the means were 15.35 to 22.64.

Two-way analysis of variance (ANOVA) was done on several variables using an EXCEL statistical package (37) to test significance of differences between treatments.

Wastewater Media

Filtered Albuquerque wastewater was enriched with TAP stock solutions nutrients (one ml each to 0.2 μm filtered liter of waste-water) and used either sterilized or unsterilized depending on the experimental design. The media were designated as ST—Sterile Wastewater enriched with 1% TAP; NST—Non-sterile Wastewater enriched with 1% TAP; WWS—Sterile Wastewater; WWNS—Nonsterile wastewater.

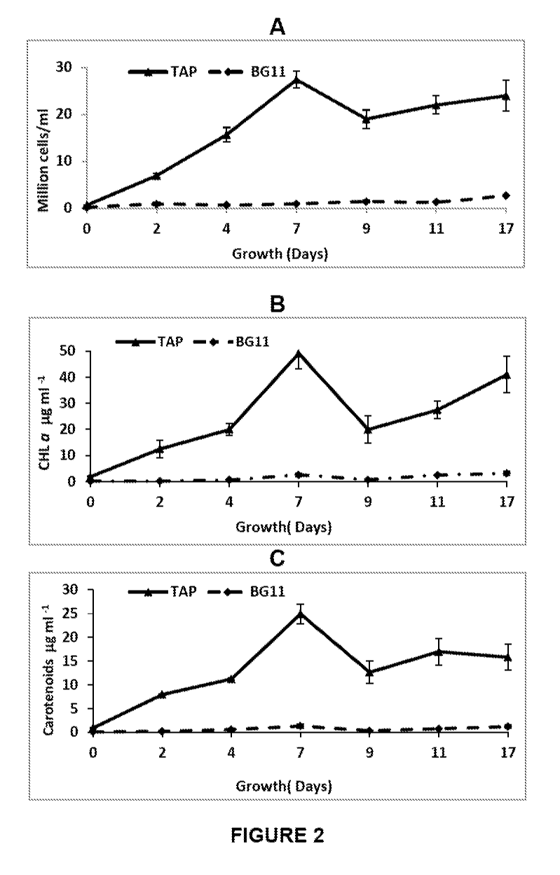

Algal cells grew readily in TAP medium and reached peak biomass levels (27.4×10⁶ cells ml⁻¹ (FIG. 2 A, Table 1), yielding 49.11 μg chlorophyll α ml⁻¹ (FIG. 2 B, Table 1) and 24.93 μg carotene ml⁻¹ on the 7^{th} day (FIG. 2 C, Table 1). Cells grew exponentially, reached a peak and subsequently decreased. Biomass levels were significantly low in BG11 medium. The division rate of cells was 0.54 day⁻¹ in TAP medium and 0.27 day⁻¹ in BG 11 (Table 1).

### Indoor Cultures in Wastewater

Cultures raised in the laboratory at 24±1° C. at continuous 132-148 μmol m⁻²s⁻¹ light in sterile wastewater enriched with 1% TAP supported good growth and yielded 10×10⁶ cells ml⁻¹, 17.6 μg chlorophyll α ml⁻¹, and 7.42 μg carotene ml⁻¹ (Table 1). The division rate was 0.24 day⁻¹ (Table 1). Growth in non-sterile wastewater, although enriched with 1% TAP, was 5.39×10⁶ cells ml⁻¹, yielding 6.79 μg chlorophyll α ml⁻¹, and 3.69 μg carotene ml⁻¹. However growth was high in unenriched sterile wastewater 10.18×10⁶ cells ml⁻¹, yielding 12.08 μg chlorophyll α ml⁻¹, and 7.64 μg carotene ml⁻¹ (Table 1), higher than in unenriched, nonsterile wastewater that has 4.33×10⁶ cells ml⁻¹, and yields 11.04 μg chlorophyll α ml⁻¹ and 5.56 μg carotene ml⁻¹ (Table 1).

Indoor wastewater cultures had more pigments per cell (range of 2.88 pg cell⁻¹ chlorophyll α to 3.43 pg cell⁻¹ chlorophyll α and 1.52 pg cell⁻¹ to 1.75 pg cell⁻¹ carotene compared to those grown either outdoors or in TAP or BG11 media (Table 1).

### Outdoor Cultures in Wastewater

Growth of cultures raised on the terrace of a building under harsh ambient conditions of light (1400-1600 watts) and temperature (˜40° C.) favorably compared to that of cultures raised indoors. The cultures raised under these harsh ambient conditions produced a biomass yielding 10.41×10⁶ cells ml⁻¹, 8.92 μg ml⁻¹ chlorophyll α, and 4.18 μg ml⁻¹ carotene (Table 1) in sterile wastewater enriched with 1% TAP; with a division rate of 0.24 day⁻¹. In unenriched sterile wastewater peak biomass was 8.81×10⁻¹ cells ml⁻¹, yielding 5.82 μgml⁻¹ chlorophyll α and 4.49 μg ml⁻¹ carotene (Table 1) with a cell division rate of 0.19 day⁻¹. Corresponding numbers for unenriched nonsterile wastewater cultures were 5.08×10⁶ cells ml⁻¹ biomass, yielding 5.41 μgml⁻¹ chlorophyll α, and 3.02 μgml⁻¹ carotene with a division rate of 0.14 day⁻¹ (Table 1).

### Analysis of Variance

Results of two-way analysis of variance (Table 2) showed that statistically significant differences existed in the biomass levels depending on the medium utilized. For example cultures grown in the defined TAP medium yielded higher levels of cells, biomass, chlorophyll α, and carotene cell⁻¹, than those in BG11 medium. Cultures grown in sterilized wastewater enriched with 1% TAP nutrients had significantly higher cell densities, chlorophyll α and carotene than those in similar media but unsterilized.

Production of biomass, i.e., cells, chlorophyll α and carotene in cultures grown indoors and outdoors in ST (Sterile medium enriched with 1% TAP), was significantly higher than in cultures grown in NST medium (non-sterile medium enriched with 1% TAP), WWS (sterile wastewater) and WWNS (nonsterile wastewater). However differences in chlorophyll α levels in cultures raised in non-sterile wastewater enriched with 1% TAP (NST) and in non-sterile wastewater (WWNS) were not statistically significant.

### Changes in Pigment Levels

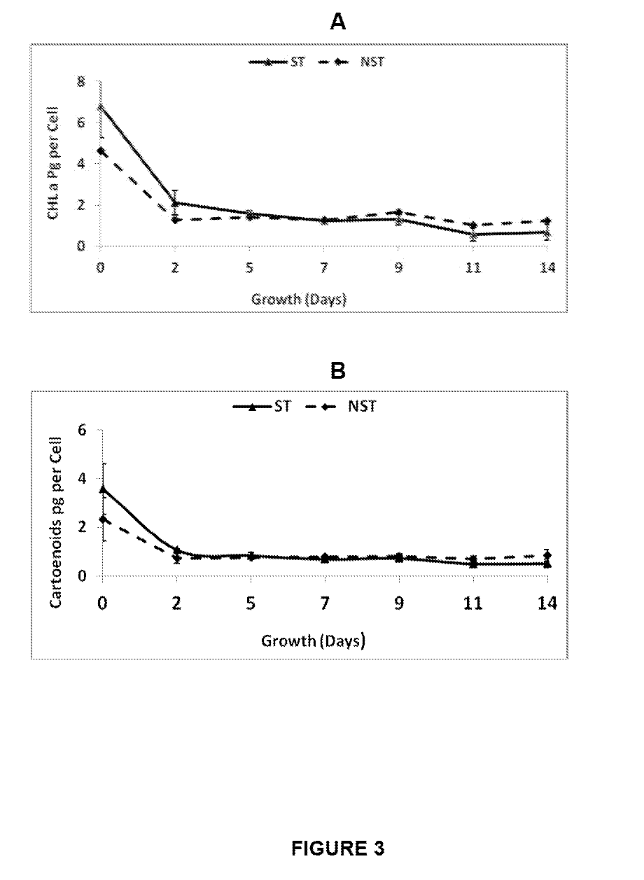

A feature of interest is the high initial levels of cellular chlorophyll α, and carotene and their gradual decrease with time (FIG. 3) in all cultures. For example cellular chlorophyll α levels (FIG. 3A) in cultures grown indoors were 0.67 pg cell⁻¹ (ST), 4.62 pg cell⁻¹ (NST), 3.26 pg cell⁻¹ (WWS) and 2.76 pg cell⁻¹ (WWNS) and the corresponding cellular carotene values were 3.58 pg cell⁻¹ (ST), 2.33 pg cell⁻¹ NST), 1.64 pg cell⁻¹ (WWS) and 1.52 pg cell⁻¹ (WWNS) (FIG. 3B).

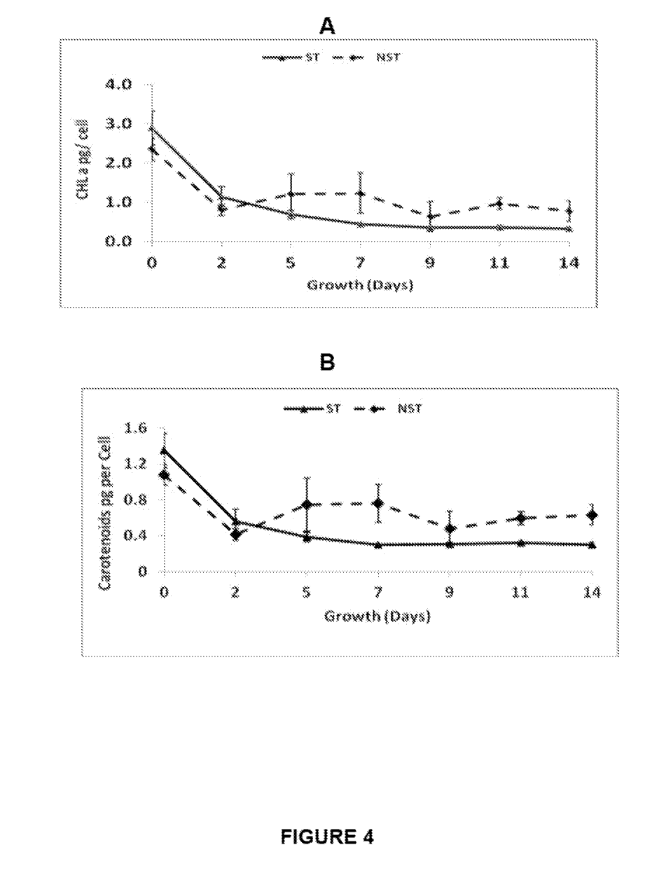

In outdoor cultures the initial cellular chlorophyll α levels (FIG. 4 A) were 2.89 pg cell⁻¹ (ST) 2.35 pg cell⁻¹ (NST), 4.83 pg cell⁻¹ (WWS) and 4.43 pg cell⁻¹ (WWNS). Corresponding carotenes were 1.35 pg cell⁻¹ (ST) 1.08 pg cell⁻¹ (NST), 0.93 pg cell⁻¹ (WWS) and 0.95 pg cell⁻¹ (WWNS). By day 14 the chlorophyll α decreased to about 14% to 85% in both indoor and outdoor cultures. Decreases in carotenes varied between 14% and 85% in indoor cultures and 22% to 63% in outdoor cultures (FIG. 4 B). Carotene also decreased and ranged from 36% to 85% in indoor cultures and 51% to 66% in outdoor cultures.

### Discussion

Our results show that microalgal extremophiles native to New Mexico can be brought into wastewater culture. Scenedesmus species Novo studied here is especially suited for mass cultivation and for utility in biotechnology. This alga is cultivable in wastewater and under the harsh ambient light and temperature conditions of semiarid regions such as Albuquerque, N. Mex. Its production is cost-effective, an important consideration in biotechnology applications. Biomass levels of our outdoor cultures were high (10.41×10⁶ cells ml⁻¹, yielding 8.92 μg chlorophyll α nil, and 4.18 μg carotene ml⁻¹), and division rates (8) compared well with those obtained on cultures raised under measurable controlled, less severe conditions of temperature and light. Harvesting the algal biomass is also simple and cost-effective as our cultured cells settle readily to the bottom and separation does not require centrifugation, flocculation, or utilization of other energy-intensive methods.

A few investigators have studied the lipid as percent dry weight of cultured algae (Table 4); our algal cells had a range of 15-85% (Table 3, Table 4) compared 0.1 to 75% reported on several species (Table 4). Several studies reported potential for sustaining algal blooms in water enriched with wastewater from municipal sewage, agriculture and industrial sources and total lipids that varied between 9 and 29% of dry weight (9). Total lipids in Chiamydomonas reinhardtii were 25.25% dry weight (10) 17.85% in Botryococcus braunii (11), 9-13.6% in Chlorella ponds enriched with dairy manure (12), and 14% to 29% in mixed algae cultures originally isolated from local wastewater treatment ponds (13). Because of their high-value for biofuel, nutraceuticals and pharmaceuticals, carotenoids and lipids from microalgae have been studied, with most investigators reporting these values as percent of cell dry weight, lipid production as mgl⁻¹d⁻¹, gl⁻¹d⁻¹, and g m⁻²d⁻¹ (1, 14, 9, 15, 16, 17, 18). Preliminary analyses of lipids on our algal slurries (Table 3) showed that lipid yield was initially high, reaching a peak (94.3 pg cell⁻¹) following 8 days of growth.

Cellular carotene in our algal cells ranged from 0.95 to 3.58 pg cell⁻¹ and compared favorably with carotene yields (Table 5) for Dunaliella salina (19) or D. salina, D. bardawil and 18 strains of microalgae isolated from tropical waters of the Bay of Bengal (20).

We have successfully brought the extremophile alga Scenedesmus species Novo, native to New Mexico; into culture.

Sequencing of the new Jemez alga was completed utilizing three different primers to completely sequence the 18S rDNA (32) and the data were used to assemble the contig. The 18S rDNA sequence of Scenedesmus species Novo [SEQ ID NO:1] is shown in the Sequence Listing at the end of this Specification.

Sequencing of the Jemez alga showed that it is most closely related to G24 (but less than 99% homologous to G4, and more distant from Scenedesmus abundans and S. communis.

In the defined TAP medium, under controlled conditions of temperature and light, high levels of biomass (cells), chlorophyll α and carotene and division rates were sustained. Further this extremophile alga grew well in wastewater under controlled conditions of temperature and light and under harsh ambient temperatures and light as well. An added advantage of our cultures is the settlement of cells readily to the bottom which makes their harvesting simple, and cost effective.

Cellular lipids in our cultures are the highest reported for microalgae. Lipids in cultures attained their peak (94.3 pg cell⁻¹) in a relatively short time, remained high and contributed between 57% and 85% of cell weight. Carotenoids were also high (0.95-3.58 pg cell⁻¹) and compared favorably with those obtained on 18 strains of microalgae isolated from the tropical waters of the Bay of Bengal.

Scenedesmus species Novo grows rapidly under harsh climatic conditions and in wastewater. Through biochemical manipulation lipid and carotene synthesis can be regulated in algae. This involves imposing a physiological stress such as nutrient starvation to channel metabolic processes towards accumulation of bioactive compounds. To enhance yield of microalgal biomass, micronutrients such as selenium, boron and iron can be optimized, along with temperature and light.

Antimicrobial Activity of Extracts of Scenedesmus novo

S. nova cells are grown at 22° C. under continuous light conditions for 10 days to achieve a dense culture. Final volume of culture is 2.0 liters. Cells are centrifuged and cell pellets are subjected to lysis using sonication in the setting of proteinase K and bath temperatures of 4° C. to avoid inactivation of proteins. Cell lysate is decanted and tested in antimicrobial screening assays against a control extract prepared from a Chlorella species.

Antimicrobial assays are conducted using turbidity assessments for Minimum Inhibitory Concentrations. Target species of bacteria include E. coli, S. aureus, K. pneumonia and P. vulgaris. In all cases, cell lysates of S. nova exhibit inhibition of growth of bacteria at 12 hours in a 96-well plate assay.

Antiparasite assays are conducted as above using 2 target organisms: Trypanosoma cruzi strain “Y” and Leishmania donovani. Cell lysates of S. novo inhibit parasite growth at 24 hours.

Antifungal assays are conducted with Candida albicans and inhibition of fungal growth in a broth assay is observed at 24 hours with cell lysates of S. novo.

All publications referred to herein are incorporated herein by reference to the extent not inconsistent herewith.

Numerical ranges mentioned herein specifically include all numbers to two decimal places that fall between the stated end points of the ranges.

It will be understood that although specific organisms, reagents, method steps and process conditions have been provided herein, equivalents of these are considered to be within the scope of the appended claims.

TABLE 1
Maximum mean values of cell numbers, chlorophyll a, and carotenoids with standard
deviations and day of attainment, in cultures of Scenedesmus sp. Novo.

| | | Cells | Chl α | Carotene | Chl α | Carotene | Chl α: | K cell |
|---|---|---|---|---|---|---|---|---|
| Growth | | 10⁶ ml⁻¹ | μg ml⁻¹ | μg ml⁻¹ | pg cell⁻¹ | pg cell⁻¹ | Carotene | div.d⁻¹ |
| 1. TAP & BG11 | TAP | 27.42 | 49.11 | 24.93 | 3.1 | 1.43 | 2.6 | 0.54 |
| | S.D | 1.83 | 6.02 | 1.3 | 0.91 | 0.4 | 0.2 | |
| | Day | 7 | 7 | 7 | 0 | 0 | 17 | |
| | BG 11 | 2.7 | 3.18 | 1.3 | 2.83 | 1.41 | 3.44 | 0.27 |
| | S.D | 0.13 | 0.88 | 0.4 | 1.17 | 0.64 | 0.43 | |
| | Day | 17 | 17 | 7 | 7 | 7 | 11 | |
| 2. Indoors | ST | 10 | 17.6 | 7.42 | 6.79 | 3.58 | 2.42 | 0.24 |
| | S.D | 0.26 | 0.73 | 1.36 | 2.22 | 1.04 | 0.46 | |
| | Day | 9 | 2 | 2 | 0 | 0 | 2 | |
| | NST | 5.39 | 6.79 | 3.69 | 4.62 | 2.33 | 2.33 | 0.25 |
| | S.D | 0.14 | 2.99 | 0.52 | 0.75 | 0.39 | 0.14 | |
| | Day | 11 | 5 | 11 | 0 | 0 | 9 | |
| | WW S | 10.18 | 12.08 | 7.64 | 3.43 | 1.75 | 2.32 | 0.15 |
| | S.D | 1.69 | 2.24 | 1.55 | 1.51 | 0.75 | 0.5 | |
| | Day | 5 | 2 | 14 | 2 | 2 | 9 | |
| | WW NS | 4.33 | 11.04 | 5.56 | 2.88 | 1.52 | 2.02 | 0.11 |
| | S.D | 0.36 | 1.51 | 0.38 | 0.68 | 0.45 | 0.12 | |
| | Day | 14 | 11 | 14 | 0 | 0 | 11 | |
| 3. Outdoors | ST | 10.41 | 8.92 | 4.18 | 2.89 | 1.35 | 2.14 | 0.24 |
| | S.D | 2.59 | 0.81 | 0.5 | 0.18 | 0.19 | 0.06 | |
| | Day | 5 | 0 | 0 | 0 | 0 | 0 | |
| | NST | 5.65 | 7.13 | 3.65 | 2.35 | 1.08 | 2.17 | 0.28 |
| | S.D | 0.54 | 0.45 | 1.63 | 0.45 | 0.11 | 0.06 | |
| | Day | 14 | 0 | 5 | 0 | 0 | 0 | |
| | WW S | 8.81 | 5.82 | 4.49 | 2.01 | 0.93 | 2.22 | 0.19 |
| | S.D | 0.38 | 2.25 | 1.64 | 0.18 | 0.05 | 0.03 | |
| | Day | 14 | 14 | 14 | 0 | 0 | 0 | |
| | WW NS | 5.08 | 5.41 | 3.02 | 2.19 | 0.95 | 2.24 | 0.14 |
| | S.D | 0.3 | 0.83 | 0.32 | 0.29 | 0.04 | 0.08 | |
| | Day | 14 | 5 | 14 | 0 | 0 | 0 | |

TABLE 2
Summary of results on two way analysis of
variance (d.f 41, F critical = 2.44).

| Growth | Source* | Variable | F value | probability | Significance |
|---|---|---|---|---|---|
| 1. Defined | TAP-BG11 | cells | 86.68 | 8.97 E-17 | Highly |
| media | | Chl α | 45.20 | 3.94 E-13 | significant |
| | | carotene | 50.89 | 8.85 E-14 | |
| | | Chl α/cell | 8.11 | 3.90 E-05 | |
| | | Carotene/cell | 5.50 | 0.0001 | |
| 2. Indoor | ST- NST | cells | 39.29 | 2.24 E-12 | |
| cultures | ST-WW S | | 55.89 | 2.68 E-14 | |
| | NST-WW NS | | 32.79 | 2.03 E-11 | |
| | WW S-WW NS | | 46.06 | 3.12 E-13 | |
| | ST- NST | Chl α | 4.68 | 0.002 | |
| | ST-WW S | | 8.459 | 2.76 E-05 | |
| | NST-WW NS | | 6.996 | 0.0001 | |
| | WW S-WW NS | | 12.05 | 1.15E-06 | |
| | ST- NST | carotene | 3.144 | 0.017 | |
| | ST-WW S | | 8.02 | 4.294 E-05 | |
| | NST-WW NS | | 11.35 | 2.03-E-06 | |
| | ST-WW NS | | 20.0 | 6.12 E-09 | |
| 3. Outdoor | ST-NST | cells | 19.11 | 1.005 E-08 | |
| cultures | ST-WW S | | 14.53 | 0.0001 | |
| | NST-WW NS | | 4.35 | 0.003 | |
| | ST-WW NS | | 87.65 | 7.75 E-17 | |
| | ST- NST | Chl α | 10.68 | 3.55 E-06 | |
| | ST-WW S | | 6.648 | 0.00002 | |
| | NST-WW NS | | 2.32 | 0.060 | Not significant |
| | ST-WW NS | | 1.93 | 0.110 | Not significant.S |
| | ST- NST | carotene | 3.37 | 0.012 | Highly significant |
| | ST-WWS | | 3.059 | 0.019 | |
| | NST-WW NS | | 3.67 | 0.008 | |
| | ST-WW NS | | 6.38 | 0.0002 | |

*ST-Sterile wastewater enriched with 1% TAP; NST-Non-sterile wastewater enriched with 1% TAP
WWS-Sterile Wastewater; WWNS -Non-sterile wastewater.

TABLE 3
Cell numbers and lipids in Scenedesmus sp Novo in
Wastewater enriched with TAP nutrients.

| Growth | Temp. | Cells | pg lipid/ | pg lipid/ | Lipid % |
|---|---|---|---|---|---|
| (Days) | and light | 10⁶/ml | cell | pg cell dry wt | cell dry wt |
| 2 | 24 ± 1° C. | 0.32 | 66.6 | 0.60 | 60 |
| | continuous | | | | |
| | 132-148 μmol | | | | |
| | photons m⁻² s⁻¹ | | | | |
| 3 | | 0.79 | 63 | 0.57 | 57 |
| 6 | | 1.97 | 76.8 | 0.69 | 69 |
| 8 | | 2.4 | 94.3 | 0.85 | 85 |
| 9 | | 2.7 | 82.5 | 0.75 | 75 |
| 2 | ~40° C. and | 0.3 | 16.7 | 0.15 | 15 |
| | Daylight | | | | |
| | 6524 -7360 μmol | | | | |
| | photons m⁻² s⁻¹ | | | | |
| 4 | | 0.62 | 20.2 | 0.18 | 18 |
| 5 | | 0.78 | 36.6 | 0.33 | 33 |
| 7 | | 0.82 | 29.2 | 0.26 | 26 |
| 9 | | 0.85 | 20.8 | 0.19 | 19 |

TABLE 4
Lipids (pg cell⁻¹) in selected microalgal cultures.

| | Growth | Lipid | Lipid pg/pg | |
|---|---|---|---|---|
| Taxa | (Days) | pg/cell | cell dry wt | Reference |
| Scenedesmus sp Nova | 1-9 | 63 to 94.3 | 0.15 to 0.85 | Present study |
| Scenedesmus sp | | | 0.06-0.184 | (18) Chen et al. 2011 |
| obliquus | | | | |
| S. obliquus | | | 0.06-0.12 | (21) Mandal and Mallick 2009 |
| S. obliquus | | | 0.128 | (22) Silva et al. 2010 |
| | | | 0.11-0.55 | (23) Gouveia and Oliveira 2009 |
| Chlorella vulgaris | | | 0.14-0.55 | (23) Gouveia and Oliveira 2009 |
| Chlorella sps. | | | 0.34-0.67 | (18) Chen et al. 2011 |
| Chlorella | | | 0.11-0.23 | (18) Chen et al. 2011 |
| prothecoides | | | | |
| Dunaliella tertiolecta | | | 0.678 | (18) Chen et al. 2011 |
| Neochloris | | | 0.35-0.65 | (23) Gouveia and Oliveira 2009 |
| oleabundans | | | | |
| N. oleabundans | | | 0.165 | (22) Silva et al. 2010 |
| Botryococcus braunii | | | 0.5 | (24) Kojima and Zhang 1999 |
| Botryococcus braunii | | | 0.25-0.75 | (1) Chisti 2007 |
| Several algae | | | 0.06-0.678 | (18) Chen et al. 2011 |
| 8 species | | | 0.05-0.63 | (16) Mata et al. 2010 |
| 21 species | | | 0.05-0.678 | (18) Chen et al. 2011 |
| 18 strains tropical | 16 | 0.22-14.77 | | (20) Keerthi pers. com |
| algae | 21 | 0.07-44.85 | | |
| D. salina | 16 | 0.21-3.45 | | |
| | 21 | 0.04-44.85 | | |
| D. bardawil | 16 | 1.25-12.78 | | |
| | 21 | 0.06-0.30 | | |
| D. tertiolecta | 16 | 0.25-1.97 | | |
| | 21 | 0.14-22.16 | | |
| D. parva | 16 | 0.76-14.77 | | |
| | 21 | 0.29-0.46 | | |
| Nannochloropsis sp. | | 0.07-0.35 | 0.02-0.04 | (15) Huerlimann et al. 2010 |
| Isochrysis sp. | | 1.16-4.93 | 0.02-0.03 | |
| Tetraselmis sp. | | 4.37-29.11 | 0.008-0.13 | |
| Rhodomonas sp. | | 0.79-12.27 | 0.001-0.017 | |
| Nannochloropsis sp | | | 0.22-0.60 | (17) Rodolphi et al. 2009 |

TABLE 5
Carotenoids (pg cell⁻¹) in selected microalgae.

| | Media NaCl | Caroten | |
|---|---|---|---|
| Alga | % | pg cell⁻¹ | Reference |
| Scenedesmus species | Fresh water | 0.95-3.58 | Present study |
| Novo | | | |
| Dunaliella salina | | 0.35-1.77 | (19) Mendoza et al. 2008 |
| Nannochloropsis | | 0.016 | (25) Forzan et al. 2007 |
| galitana | | | |
| Haematococcus | | 25 | (26) Cifuentes et al. 2003 |
| pluvialis | | | |
| | N₂ normal | 8-15 | |
| | N₂ deprived | 10.3-25 | |
| 18 strains of | | 0.24 to 4.75 | (20) Keerthi pers. com |
| microalgae | | | |
| Dunaliella bardawil | 10 | 0.67-27.53 | (20) Keerthi pers. com |
| | 12.5 | 0.49-2.07 | |
| | 15 | 0.32-14.07 | |
| | 20 | 1.67-3.79 | |
| | 25 | 0.61-7.92 | |
| | 30 | 0.57-7.28 | |
| D. salina | 10 | 0.3-1.61 | (20) Keerthi pers. com |
| | 12.5 | 0.3-1.89 | |
| | 15 | 0.34-1.69 | |
| | 20 | 0.36-1.77 | |
| | 25 | 0.38-1.85 | |
| | 30 | 0.27-1.61 | |
| D. salina | | 1.1-2.80 | (27) Pisal and Lele 2005 |

### REFERENCES

1. Chisti Y (2007) Biodiesel from microalgae. Biotechnol Adv 25: 294-306.
2. Haag A L (2007) Algae Bloom again. Nature 447: 520-521.
3. Weyer K M, et al. (2010) Theoretical maximum Algal Oil Production. Bioenerg. Res. 3: 204-213.
4. International Energy Agency Report http://www.iea.org/journalists/index.asp
5. Alabi A O (2009) Microalgal technologies and processes for biofuels/bioenergy production in British Columbia. The British Columbia Innovation Council 1-74 pp.
6. Liska A J, et al. (2004) Enhanced Photosynthesis and Redox Energy Production Contribute to Salinity Tolerance in Dunaliella as Revealed by Homology-Based Proteomics. Plant Physiology 136:2806-2817.
7. Subba Rao D V, et al. (2005) Growth and photosynthetic rates of Chlamydomonas plethora and Nitzschia frustula cultures isolated from Kuwait Bay, Arabian Gulf, and their potential as live algal food for tropical mariculture. Marine Ecology, 26: 63-71.
8. Subba Rao D V (2009) Cultivation, Growth Media, Division rates and applications of Dunaliella species. Pp 45-90. In: Ben-Amotz A. Polle J E W, Subba Rao D V, editors. Alga Dunaliella Biodiversity, Physiology, Genomics and biotechnology. Enfield: Science Publishers: 2009.
9. Pittman J K, et al. (2010) The potential of sustainable algal biofuel production using wastewater resources. Bioresource Technology 102: 17-25.
10. Kong Q X, et al. (2010) Culture of microalgae Chlamydomonas reinhardtii in wastewater for biomass feedstock production, Appl. Biochem. Biotechnol. 160: 9-18.
11. Orpez R, et al. (2009) Growth of the microalga Botryococcus braunii in secondarily treated sewage. Desalination 246: 625-630.
12. Wang Y C, et al. (2010) Anaerobic digested dairy manure as a nutrient supplement for cultivation of oil-rich green microalgae Chlorella sp. Bioresour. Technol. 101: 2623-2628.
13. Woertz I, et al. (2009) Algae Grown on Dairy and Municipal Wastewater for Simultaneous Nutrient Removal and Lipid Production for Biofuel Feedstock. Jour. Envi. Eng.© ASCE I November 135: 1115-1122.
14. Harun R, et al. (2010) Bioprocess engineering of microalgae to produce a variety of consumer products. Renewable and Sustained Energy reviews 14: 1037-47.
15. Huerlimann R, et al. (2010) Growth: lipid content: productivity, and fatty acid composition of tropical microalgae for scale-up production. Biofuels and Environmental Biotechnology DOI 10. 1002/bit.22809
16. Mata T M, et al. (2010) Microalgae for biodiesel production and other applications: A review. Renewable and Sustainable Energy reviews. 14: 217-32.
17. Rodolfi L, et al. (2008). Microalgae for Oil: Strain selection: induction of lipid synthesis and outdoor mass cultivation in a low-cost photobioreactor, Biotechnology and Bioengineering. 102: 100-112.
18. Chen C V, et al. (2011). Cultivation, photobioreactor design and harvesting of microalgae for biodiesel production: A critical review. Bioresource Technology 102: 71-81.
19. Mendoza H, et al. (2008) Characterization of Dunaliella salina strains by flow cytometry: a new approach to select carotenoid hyperproducing strains Electronic Journal of Biotechnology ISSN: 0717-3458, 11: 2-13.
20. Keerthi et al. 2012 personal communication
21. Mandal 5, Mallick N (2009) Microalga Scenedesmus obliquus as a potential source for biodiesel production. Appl Microbiol Biotechnol 84: 281-91.
22. Silva T L, et al. (2010) Oil Production Towards Biofuel from Autotrophic Microalgae Semicontinuous Cultivations Monitorized by Flow Cytometry. Applied Biochemistry and Biotechnologyl 59: 568-578, DOI: 10.1007/s12010-008-8443-5.
23. Gouveia L, Olievera A C (2009) Microalgae as a raw material for biofuel production. Journal of Industrial Microbiology and Biotechnology. 36: 269-74. DOI:10. 1007/s 10295-008-0495-6.
24. Kojima E: Zhang K (1999) Growth and hydrocarbon production of microalga Botryococcus braunii in bubble column photobioreactors. Journal of Bioscience and Bioengineering: 811-815.
25. Forjan E, et al. (2007) Enhancement of carotenoid production in Nannochloropsis by phosphate and sulphur limitation. pp 356-364 in Communicating Current Research and Educational Topics and Trends in Applied Microbiology. (Ed). A. Méndez-Vilas.
26. Cifuentes A S, et al. (2003) Optimization of biomass, total carotenoids and astaxanthin production in Haematococcus pluvialis, Flotow strain Steptoe (Nevada, USA) under laboratory conditions. Biol. Res. 3 6: 343-357.
27. Pisal D S, Lele S S (2005) Carotenoid production from microalga, Dunaliella salina. Ind Jour Biotech. 4: 476-483.
28. Guillard R R L, Ryther J H (1962) Studies of marine planktonic diatoms. I. Cyclotella nana Hustedt and Detonula confervacea Cleve. Can. J. Microbiol. 8: 229-239.
29. Stanier R Y, et al. (1971) Purification and properties of unicellular bluegreen algae (Order Chroococcales). Bacteriol. Rev. 35: 171-205.
30. Gorman D S, Levine R P (1965) Cytochrome f and plastocyanin: Their sequence in the photosynthetic electron transport chain of Chlamydomonas reinhardttii. Proc. Natl. Acad. Sci. USA 5: 1665-1669.
31. Hutner S H, et al. (1950) Some approaches to the study of the role of metals in the metabolism of microorganisms. Proc. Am. Philos. Soc. 94: 152-170.
32. GenBank: HQ900842.1
33. Furnas M (2002) Measuring the growth rates of phytoplankton in natural populations. In: Subba Rao D. V. (Ed), Pelagic Ecology Methodology. A. A. Balkema Publishers Lisse/Tokyo, 221-249.
34. Jeffrey S W, Humphrey G F (1975) New spectrophotometric equations for determining chlorophylls a, b, c₁ and c₂ in higher plants, algae and natural phytoplankton. Biochem. Physiol. Pflanzen. 167: 191-194.
35. Borowitzka M J, Siva C J (2007) The taxonomy of the genus Dunaliella (Chlorophyta, Dunaliellales) with emphasis on the marine and halophilic species. Journal of Applied Phycology 19: 567-590.
36. www.AvantiLipidscom
37. Dytham C (1999) Choosing and using Statistics: A Biologist's Guide. Blackwell Sciences. 218 pp.
38. Nold. S. C., et al. (2010), “Eukaryal and archaeal diversity in a submerged sinkhole ecosystem influenced by sulfer-rich, hypoxic groundwater,” Great Lakes Res. 36, 366-375, GenBank Accession No. EU910612.

1. An isolated and purified microalga Scenedesmus species Novo obtained from an alga collected at latitude 35.769 and longitude 106.692, or progeny thereof, capable in culture including TAP medium of producing a biomass of about 10.41×10⁶ cells per ml and at least about 4 μg per ml of carotene under outdoor growth conditions comprising temperatures reaching 40° C. or higher.

2. The microalga of claim 1 having an 18S ribosomal RNA gene sequence at least about 99% to about 100% identical to SEQ ID NO:1

3. A culture of the isolated and purified microalga of claim 1 cultivated in nutrient-enriched wastewater.

4. The culture of claim 3 cultivated at room temperature.

5. A culture of the microalga of claim 1 cultivated in nutrient-enriched wastewater having an average lipid content of between about 63% and about 95% of the dry weight of the cells.

6. A culture of the microalga of claim 1 having a lipid content of between about 16.7 and 81.4 pg per cell.

7. A culture of the microalga of claim 1 cultivated in water comprising TAP medium capable of producing a chlorophyll α content of between about 20 and about 49 μg per ml.

8. A culture of the microalga of claim 1 cultivated in water comprising TAP medium capable of producing a carotene content of about 10 to about 25 μg per ml.

9. A culture of the microalga of claim 1 cultivated in wastewater at temperatures of at least about 40° C. to about 100° C.

10. The culture of claim 9 cultivated in a nutrient-enriched medium capable of producing an average lipid content between about 15% to about 74% based on dry weight of the cells.

11. The culture of claim 9 capable of producing a chlorophyll α content of at least about 5.8 μg per ml.

12. A culture of the micralga of claim 1 that has circular single cells and/or clumps of cells of up to about 360 cells.

13. A dried algal biomass produced by drying microalgae of claim 1, said dried algal biomass having less than about 5% water content.

14. A method for culturing and harvesting extremophilic microalgae comprising:
preparing a growth composition comprising said extremophilic microalgae and water comprising nutrients capable of enhancing growth of said microalgae;
allowing said microalgae to proliferate in said composition at room temperature or under ambient outdoor conditions comprising intervals of ambient temperatures of at least about 40° C. and ambient light of up to about 1400 to about 1600 watts;
dewatering said composition and recovering an algal biomass comprising said microalgae and less than about 5% water content.

15. The method of claim 14 wherein said dewatering is performed in an AlgaeVenture Systems micro-Solid-Liquid-Separation System.

16. The method of claim 14 wherein said extremophilic microalgae is Scenedesmus species Novo.

17. The method of claim 14 wherein said growth composition comprises Scenedesmus species Novo and wastewater.

18. The method of claim 14 wherein said nutrients suitable for enhancing growth are selected from the group consisting of TAP medium components, selenium, boron and iron.

19. A method of inhibiting growth of a microorganism comprising contacting cells of said microorganism with an extract of Scenedesmus species Novo.

20. The method of claim 19 wherein said microorganism is selected from the group consisting of bacteria, viruses, parasites, and fungi.
